(19) United States
(12) Patent Application Publication
(10) Pub. No.: US 2010/0008820 A1
(43) Pub. Date: Jan. 14, 2010

(54) PROCEDURE AND APPARATUS FOR CLEANING OF GAS, LIKE AIR FROM UNWANTED GASEOUS COMPOUNDS

(75) Inventor: Heikki Savikko, Turku (FI)

Correspondence Address: BIRCH STEWART KOLASCH & BIRCH, PO BOX 747, FALLS CHURCH, VA, 22040-0747, US

(73) Assignee: ODOROFF OY, Piispanristi (FI)

(21) Appl. No.: 12/303,081

(22) PCT Filed: Jun. 2, 2006
(86) PCT No.: PCT/FI2006/050228

§ 371 Date: Jun. 25, 2009

(51) Int. Cl. A61L 9/00; G05D 9/00; B01D 53/14

(52) U.S. Cl. 422/4; 422/106

(57) ABSTRACT

This invention relates to a method and apparatus for purifying gases such as air of unwanted gaseous compounds, in which method the gas to be purified is brought into contact with a solution (2) that binds the unwanted gaseous compounds, and in which method the gas to be purified is caused to flow through a reactor filler (3) in a space filled with the solution that binds the unwanted gaseous compounds. At least the saturation level of the solution (2) and the surface level of the solution (2) are monitored in essence continuously, and on the basis of monitoring data the solution's saturation level and surface level are maintained essentially within predetermined limit values.

This invention relates to a method for purifying gases such as air of unwanted gaseous compounds, as described in the preamble of claim 1, and to a purification apparatus as described in the preamble of claim 6.

Removing malignant odours from air has proven to be very difficult because most cases contain a complex series of compounds with a varying odour threshold, which can be very low for some compounds. Some published studies have analysed more than one hundred compounds present for example in rubbish dump smells and found that certain compounds have odour thresholds in the region of 1 ppb (part per billion). In other words, the odours felt by human noses come from very small quantities of scent.

A typical unpleasant odour compound is hydrogen sulphide. Its odour threshold is around 0.5 ppm (parts per million). An odour compound that is often present around pulp industry plants is methyl mercaptan, whose odour threshold is less than one part per billion.

Eliminating odours from air could be described as the process of removing very small amounts of organic odour compounds from large quantities of air.

Solutions for removing unpleasant smells from air have long been sought. Perhaps the oldest known method is burning odorous air. In this known method, the temperature of the air is raised to around 600-800° C., which breaks down all organic compounds and removes odours from the air. In practice, however, the method has not become established because it is financially unviable. It requires large amounts of air to be heated up, after which the heat should be recovered. Air has poor heat transfer properties, however, which makes the equipment large and expensive. Extensive research has been conducted into biofilters, and some such filters have been used in practice. Biofilters are generally large box-shaped devices filled with bark, peat or a similar material. A bacterial strain that builds slime is implanted in the medium's surface in order to “consume” some of the compounds—particularly hydrogen sulphide—present in odorous air. These bacteria are less capable of removing nitrogen compounds, so generally the end result is some degree of reduction of the odour intensity. Biological events take a long time, so the processes are slow and the equipment is very large. Another problem arises from achieving a large enough surface of contact between odorous air and the bioactive material. The larger the size, the more contact between air and the medium can be achieved in biofilters.

Strong oxidants have also been used to reduce the odour intensity of air; an example is ozone gas. There are apparatuses that generate ozone gas using an electrical current and then blow it out into the odorous air current. The problem is that only some odour compounds oxidise, while others oxidise with more difficulty. Another practical problem is how to feed the ozone flow into a large quantity of odorous air so that contact (i.e. mixing) is even. Further restrictions are placed by the fact that the ozone content of air permitted by safety regulations is very low. In reality, only a degree of reduction of odour intensity can be achieved using ozone.

Gas washers are used mainly to remove a specific compound from an air current. A typical example is the removal of sulphur dioxide from air in industrial processes. In a gas washer, air is usually fed through a vertical reactor filled with a porous medium, while a suitable chemical solution that reacts with sulphur dioxide flows in the opposite direction. The method is suitable for situations where the concentration of a single compound must be reduced for example from 10,000 ppm to 10 ppm. The method is not very well suited to cases where there is a highly varied group of compounds, whose concentrations should be reduced from around 5 ppm to around 0.1 ppm. In washers it is difficult to achieve sufficiently effective contact between the chemical and the passing air. The effectiveness of contact can be improved by adding multiple washes to the process, but this increases the cost proportionally. One improved gas washer solution is described in Finnish patent application no. 20031728, in which, among other processes, the gas is broken down into microbubbles before being fed into the chemical solution. Microbubbles mean air bubbles within the solution, with a diameter equal to or less than one millimetre. In addition, the surface of contact between the gas bubbles and the chemical solution is increased by placing a suitable filler medium within the reactor.

A so-called dry washer technique is also well known. In this method, an inert, porous medium is treated with a strong oxidiser such as potassium permanganate. When odorous air is blown through the porous material, it oxidises all oxidisable compounds, mainly hydrogen sulphide, thus reducing the odour intensity of the outflow air. In order for the oxidisation reactions to be complete enough, several overlapping phases must be used, which increases the cost of the process. Two factors increase costs in this process, because to achieve sufficient contact between the reactive solid and the flowing gas, the contact surface must be increased by having multiple stages; this increases the flow resistance, which in turn increases the electricity consumption.

Activated carbon filters are also used to deodorise air. In these, certain sulphurous compounds stick to (i.e. are absorbed by) the surface of the carbon. These filters are used particularly for removing hydrogen sulphide. However, they are inadequate for removing all odours.

A further problem is the fact that all air deodorisation events are always characterised by significant fluctuations in odour intensity. For instance in residential areas, the odour problems caused by the sewer network are severe in the morning, before people leave for work; then they improve during the day and worsen again in the evening, once people have returned home. Fully automatic equipment should be able to adjust its purification capacity in accordance with these fluctuations, as optimally and cost efficiently as possible. None of the abovementioned solutions has addressed the problems caused by such fluctuations in odour intensity.

Below, the method and apparatus according to the invention will be known collectively as “the solution according to the invention”. The object of this invention is to create a solution for removing undesirable gaseous compounds that allows the purification of gases such as air in such a way as to overcome the limitations listed for the abovementioned methods. The aim is to purify air to such an extent that, using the human sense of smell as a gauge, the out flowing air is essentially free of odour-causing compounds, and to create a financially viable method. The object of the invention is also to control the purification process automatically and continuously so that the purification capacity remains sufficient at all concentrations of odorous gases. The aim is also to achieve an apparatus that automatically replaces the odour binding solution when it becomes saturated and loses its purification ability.

The method according to the invention is characterised by what is disclosed in the characterisation part of claim 1, and the apparatus according to the invention is characterised by what is disclosed in the characterisation part of claim 6. Other favourable embodiments of the invention are characterised by what is disclosed in the other claims.

The solution according to the invention has one or more of the following benefits:
The solution is more effective than any known solution. Using the human sense of smell as a gauge, the result is essentially perfect.
The solution is financially viable, because the structures required are simple and can make use of industrial waste.
Thanks to the simplicity of the functions, the solution is reliable and requires very little maintenance.
The solution is very safe for users.
The solution is automatic. It adapts to fluctuations in odour intensity throughout the day and can automatically replace the binding solution when it is saturated.
The solution is environmentally friendly. Despite the fan, under operation the solution's reactor is not noisy, because it can very easily be soundproofed.
Because the process works at essentially low pressure, the structures can be built for instance of thin-walled plastic, which lowers costs.

Below, the invention is described in detail using an application example of the solution according to the invention, by referring to the appended figures, in which

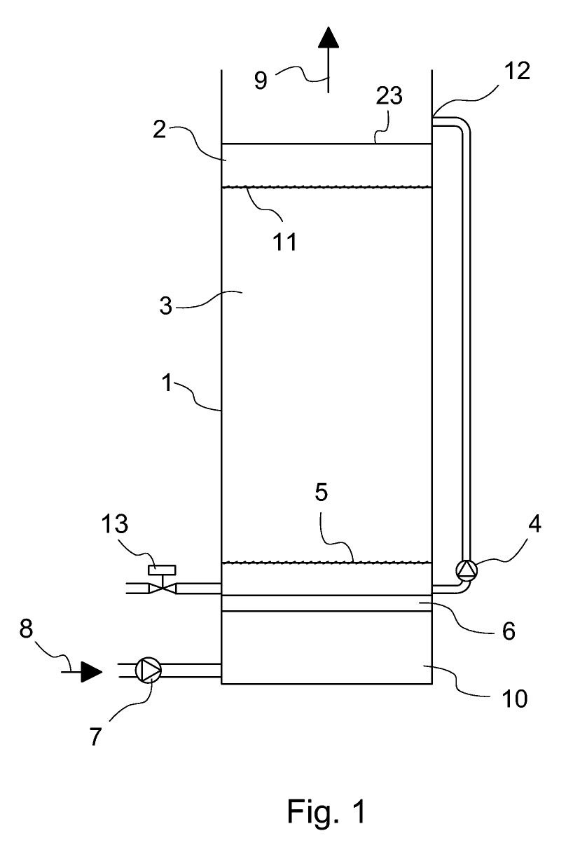

FIG. 1 shows an apparatus according to known technology in simplified and diagrammatic form, viewed from the side, and

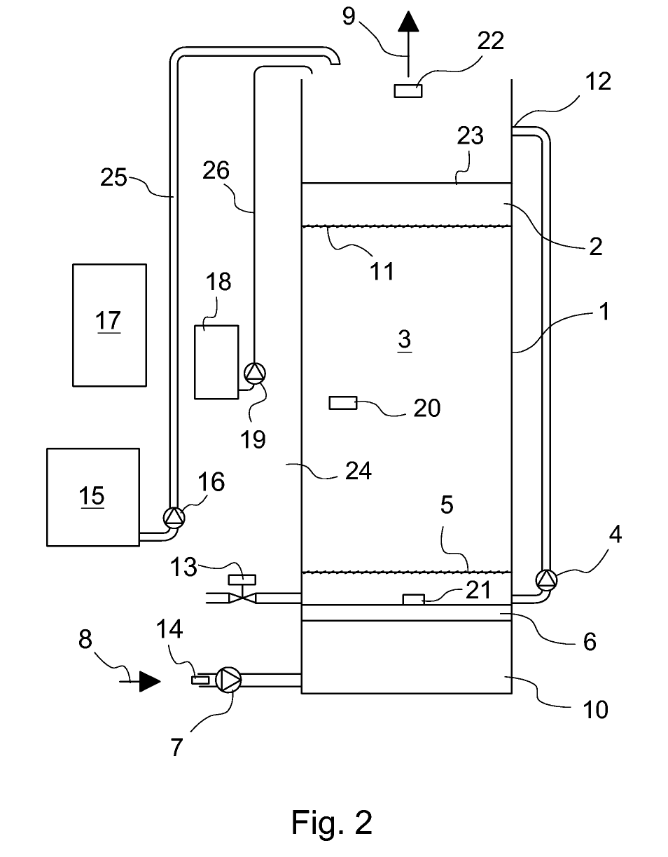

FIG. 2 shows an apparatus according to the invention in simplified and diagrammatic form, viewed from the side.

The apparatus according to known technology shown in FIG. 1 and the apparatus according to the invention shown in FIG. 2 contain at least an absorption reactor, which consists at least of an inlet 8 for the air to be purified, of a reactor space 1, filled up to the determined maximum level 23 with liquid chemical solution, here known as binder solution 2, and with reactor filler 3 that affects the passage of the air, and of an outlet 9 for the purified air.

Below the reactor space 1, at the base of the reactor and immediately after the air inlet 8, the apparatus has a space 10 for equalising the air flow, designed to create even pressure across the entire base of the reactor. In addition, next to the air inlet 8, the lower part of the apparatus contains a fan 7, which is designed to push the air to be purified into the absorption reactor's reactor space 1. Moving upwards, i.e. in the direction of flow of the air, the next element following the air equalising space 10 is an air atomiser element 6, such as a superoxidant film or a similar porous material, to atomise the air. This device is characterised by containing thousands of small holes or porous channels per square decimetre. The holes or channels are so small that the binder solution 2 cannot permeate them and pass to the area below the atomiser element 6. When passing through the atomiser element 6, the air to be purified is broken down into large quantities of small gas bubbles that begin to rise through the binder solution 2 that fills the reactor.

The abovementioned reactor filler 3 is designed to be supported by the top surface of a steel base mesh 5 placed in the lower part of the reactor space 1, which base mesh 5 is at a certain distance above the atomiser element 6 such that there is a space free of reactor filler 3, filled with binder solution 2, between the base mesh 5 and the atomiser element 6. The reactor filler 3 consists for example of metal shavings, most favourably stainless steel shavings. This flexible material has a density of 100-120 kg/m³ upon formation. The reactor filler 3 is pressed in between a top mesh 11 that acts as a top reinforcement and the base mesh 5, with a density of 100-400 kg/m³. The higher the density, the more effectively the filler breaks down the gas bubbles coming from below. The higher the flow resistance of the rising air current, the higher the pressure that is needed for achieving the air bubble flow, which increases the power consumption of the fan 7.

To make the process more effective, the binder solution 2 can be made to flow through the abovementioned reactor filler 3, which causes the binder solution to flow in a current containing random small vortices. When a gas bubble current is pushed upwards through the binder solution 2 in this kind of flow, the chances of the gas molecules coming into contact with the binder solution 2 are increased. In the solution according to the invention, this beneficial added effectiveness feature is organised by placing a circulation pump 4 to pump the binder solution 2 from the lower part of the reactor space 1, below the reactor filler 3 and immediately above the air atomising element 6, to section 12 in the upper part of the reactor, above the top surface 23 of the binder solution 2. This causes a top-to-bottom flow of the binder solution 2 in the reactor space 1.

The binder solution batch placed in the reactor space 1 becomes saturated after a certain time. It must then be replaced. For this purpose, the lower part of the reactor space 1, above the atomiser element 6, has a drainage valve 13, which is opened to cause the saturated binder solution 2 to flow gravitationally out of the reactor.

The apparatus according to the invention displayed in FIG. 2 differs from the apparatus according to known technology displayed in FIG. 1 in that the apparatus according to the invention is equipped to automatically monitor changes in the odour intensity of the air to be purified and react to it so that the purification capacity remains essentially the same in all circumstances. In addition the apparatus according to the invention is equipped to automatically monitor the quantity and saturation level of the binder solution and to add solution or replace the solution when necessary.

To complete the abovementioned automatic functions, the reactor is equipped with at least one programmable regulating element 17, with sensors 14, 20, 21 and 22 connected to the regulating element 17, with actuators such as pumps 16 and 19, with a water tank 15 also connected to the regulating element 17, as well as with a storage tank 18 containing binder solution concentrate. In addition, at least the fan 7, the drainage valve 13 and the circulation pump 4 are designed to be controlled by the regulating element 17. The programmable regulating element 17 contains at least communicating equipment, recording equipment, processing equipment and an interface. The necessary limit value data and other data related to controlling the purification process are stored in the regulating element 17, and the regulating element 17 is equipped to independently process the measurement data received from the sensors 14, 20, 21 and 22, and to independently control the process by controlling the actuators 4, 7, 13, 16 and 19 on the basis of these measurement data.

A pressure sensor 14 is connected to the air inlet 8 before the fan 7, to measure any changes in pressure in the incoming air. Such pressure changes can take place for instance in cases where the reactor is placed in enclosed spaces such as a sewage pumping station. As the water level rises in the well, the air pressure rises above the general external air pressure; conversely, as the well empties out, the air pressure falls below the general external air pressure. When the pressure has fallen enough, fresh air begins to flow into the surrounding space, so the reactor is no longer needed. Sensor 14 informs the regulating element 17 of pressure changes, and the regulating element contains the necessary equipment for reacting to the pressure change by controlling the fan 7. When the pressure falls below a predetermined limit value, the regulating element 17 stops the fan 7; once the pressure has risen again above a predetermined limit, the regulating element 17 restarts the fan 7 automatically.

The reactor space 1 also contains an electrical conductivity sensor 20, designed to measure the saturation level of the binder solution 2 as changes in conductivity and to report the saturation level of the solution to the regulating element 17. When the saturation level falls below a predetermined reading stored in regulating element 17, the regulating element 17 causes the drainage valve 13 to open, which causes the binder solution 2 to flow gravitationally out of the reactor. Similarly, there is a surface level sensor 21, designed to monitor the surface level of the binder solution 2 and to inform the regulating element 17 when the binder solution has drained completely out of the reactor 1. After this, the regulating element 17 starts up the pump 16 in the water tank 15, to pump water into the reactor space 1 through a pipe 25 and via the open top part of the reactor space 1. The surface level sensor 21 constantly measures the level of liquid, and the reactor space 1 fills on the basis of a signal given by the surface level sensor 21 up to the limit that the regulating element 17 has calculated as the appropriate level for achieving the correct concentration. After this, the regulating element 17 stops the pump 16 in the water tank 15 and starts up the pump 19 in the storage tank 18, in order to pump concentrated chemical solution in to the reactor space 1 through a pipe 26, until the surface level sensor 21 signals that the correct surface level for the desired concentration has been reached. After this, the reactor is again ready to operate, so the regulating element 17 stops pump 16 and starts up the fan 7.

The surface level sensor 21 can for example be a pressure sensor placed at the bottom of the reactor space 1, below the base mesh 5, to measure the pressure of the liquid above it.

The reactor also contains an odour sensor 22, designed to react to changes in the concentration of scent in the air, informing the regulating element 17. The odour sensor 22 is placed at the air outlet 9 from the reactor to measure the concentration of scent in the purified air. Three different action alternatives are programmed into the regulating element 17 as reactions to rising odour concentrations in the outflowing air. One alternative is that the regulating element 17 adjusts the pump 19 in the storage tank 18 to pump more concentrated binder solution into the reactor space 1, to increase the concentration of binder solution 2. This can only be done up to a certain maximum level, however. If this does not reduce the scent concentration sufficiently, the regulating element 17 starts up the circulation pump 4, which causes an internal counter-flow by recirculating the binder solution 2 so that it flows downwards through the reactor space 1, increasing the purification efficiency of the reactor. If the scent concentration in the outgoing air is still too high, the regulating element 17 reduces the amount of incoming air by reducing the rotation speed of the fan 7.

The liquid surface of the binder solution 2 may fall within the reactor for instance due to drop leakage or evaporation caused by high temperatures. A specific lower limit is programmed into the regulating element 7 to address this. When the surface level sensor 21 informs the regulating element 17 that the surface of the binder solution 2 has fallen to the lower limit, the regulating element 17 calculates the amount of water and chemical solution needed and starts up pumps 16 and 18 to raise the level of the binder solution 2 to the desired level. When the surface has reached the desired level, the regulating element 17 receives a signal from the surface level sensor 21 and consequently stops pumps 16 and 18.

The main prerequisite for the removal of very small concentrations of odorous compounds from air is achieving as complete a contact as possible between the odour-binding element—in this case the binder solution 2—and the odorous compounds. It is essential to the method according to the invention that in the purification process, the odorous air is put into contact with the binder solution 2 in a way that allows almost complete contact between the binder solution and the odorous compounds. In the method according to the invention, the incoming air is first broken down using so-called superoxidant films 6. This stage breaks the air down into large amounts of small gas bubbles. After that the atomised air is conducted into contact with the reactor filler 3, whose properties are such that it breaks the air bubbles into still smaller bubbles and activates a random motion of the bubble current. The packed metal shavings acting as reactor filler 3 have a large surface area in relation to their volume and direct the gas bubbles randomly. Due to their sharp edges, they cut and break down the gas bubbles. In one embodiment of the method, the amount of contact between the gas bubbles and the binder solution is increased by causing a random, turbulent motion in the binder solution as it flows against the current through the reactor filler 3.

In addition, in the method according to the invention, the purification process is controlled automatically and continuously such that the saturation level and the surface level of the solution 2 are monitored in essence continuously, and that at least the saturation level and the surface level are maintained within predetermined limit values on the basis of monitoring data. Similarly, the odour intensity of the outgoing, purified air is monitored in essence continuously. When the odour intensity exceeds a predetermined limit value, automatic measures are taken to lower the odour intensity to an acceptable level.

Through these measures, the purification capacity is maintained in all concentrations of odorous gases. At the same time, the odour-biding solution 2 is replaced automatically when it becomes saturated and loses its purification ability. The automatic controlling of the purification process takes place at least with a regulating element 7 and with sensors 14, 20-22 and actuators 4, 7, 13, 16, 18, all connected to the regulating element 7.

The main operating principle of the invention is to create as complete a contact as possible between the odorous air and the active binder solution 2, in order to allow almost all the scent molecules in the air to come into contact with the surface of the binder solution 2 and to be absorbed into the binder solution and thus be transferred into the liquid phase. This should take place as quickly as possible, in order to keep the size of the equipment small in relation to the amount of air flowing to it. The process according to the invention requires the reactions between the scent compounds and the binder solution 2 to be sufficiently fast to avoid creating a bottleneck in the process. There are several alternatives in known principles of organic chemistry. In addition, the main operating principle relates to the automatic regulation of the equipment to respond to changes in the amount of odorous gases and in the saturation level of the binder solution 2.

The solution according to the invention is characterised by being freely adjustable to any amount of air flow. As the air flow increases, the diameter of the reactor and the size of its atomising elements must grow.

Those skilled in the art will clearly see that the invention is not limited to the example given above, but can be varied within the scope of the patent claims given below. Thus, other materials, such as shavings from plastic lathing, crushed rock material such as clay aggregate, or other suitable reactor fillers can be used instead of steel shavings.

Those skilled in the art will also clearly see that any so-called superoxidant film that forms microbubbles in the binder solution is suitable for use in the first-stage atomising element. Ceramic materials characterised by the large proportion of pore channels in relation to their surface area can also be used instead of superoxidants.

Further, those skilled in the art will see that the location, number and operating principles of the sensors mentioned can vary. For example, other kinds of surface level sensors can be used instead of a pressure sensor.

Further, those skilled in the art will see that the uses of the invention can vary and that it can be applicable for example to: removing poisonous gases from air; removing general impurities from air; removing odours from breathing air. As the air passes through a suitable liquid, most of the particles suspended in it are transferred to the liquid. claims 1-12. (canceled)

13. A method for purifying gases such as air of unwanted gaseous compounds, in which method the gas to be purified is brought into contact with a solution (2) that binds the unwanted gaseous compounds, and in which method the gas to be purified is caused to flow through the solution (2) and through inside the solution (2) locating reactor filler (3) in a space filled with the solution (2) that binds the unwanted gaseous compounds, characterised in that at least the surface level of the solution (2) through which the gas to be purified is caused to flow is monitored in essence continuously, and in that the monitoring data is used to maintain at least surface level within predetermined limit values.

14. A method according to claim 13, characterised in that the odour intensity of the outgoing, purified air is monitored in essence continuously, and when the odour intensity exceeds a predetermined limit value, automatic measures are taken to lower the odour intensity to an acceptable level.

15. A method according to claims 13 or 14, characterised in that the method monitors changes in pressure taking place at the air inlet such that when the pressure drops below a predetermined limit value, the fan (7) that blows air into the reactor is stopped, and when the pressure rises above a predetermined limit value, the fan (7) is started automatically.

16. A method according to any of claims above, characterised in that the purification process is monitored and controlled in essence continuously with the use of sensors (14, 20-22), a regulating element (17) and actuators (4, 7, 13, 16, 19) connected to the reactor, such that readings received from one or more sensors (14, 20-22) are conducted to the regulating element (17), where they are processed, on the basis of which processing the regulating element (17) controls the necessary actuator(s) (4, 7, 13, 16, 19), until a predetermined state is achieved.

17. A method according to any of claims above, characterised in that the gas to be purified is broken down using a gas flow atomising element (6) into essentially small micro-bubbles, and in that the solution (2) that binds the unwanted gaseous compounds is caused to flow in the reactor space (1) in essentially the opposite direction from the gas flow consisting of microbubbles, and in that the micro-bubbles are caused to collide in the reactor space (1) with metal shavings or similar elements forming a reactor filler (3) that cuts the microbubbles and causes random vortices.

18. An apparatus for purifying gas such as air of unwanted gaseous compounds, which apparatus consists at least of an inlet (8) for the gas to be purified, a reactor space (1) and a reactor filler (3), arranged in the reactor space (1) so as to be surrounded by a solution (2) that binds unwanted gaseous compounds, and a fan (7) or similar device causing the gas to be purified to flow through the reactor filler (3) and the solution (2) in the reactor space (1), characterised in that the apparatus contains at least a sensor (21) for measuring the surface level of the binding solution (2) through which the gas to be purified is caused to flow, and at least one regulating element (17), designed to control the purification process on the basis of measurement data provided by at least the sensor (21).

19. An apparatus according to claim 18, characterised in that the sensor (21) is designed to monitor the purification process in essence continually, and the sensor's (21) measurement data is designed to be conducted to the regulating element (17) for processing.

20. An apparatus according to claim 18 or 19, characterised in that the apparatus contains a sensor (22) for measuring the odour intensity of the purified air, which sensor (22) is designed to monitor the purification process in essence continually, and which sensor's measurement data are designed to be conducted to the regulating element (17) for processing.

21. An apparatus according to claims 18, 19 or 20, characterised in that the apparatus contains, connected to the air inlet, a sensor (14) for measuring changes in pressure, which sensor (14) is designed to measure pressure changes in essence continually, and which sensor's (14) measurement data are designed to be conducted to the regulating element (17) for processing.

22. An apparatus according to any of the claims 18-21, characterised in that the apparatus contains one or more actuators (4, 7, 13, 16, 19) designed to be controlled by the regulating element (17), and in that the purification process is designed to be monitored and controlled in essence continually with the use of one or more sensors (14, 20-22), a regulating element (17) and one or more actuators (4, 7, 13, 16, 19) connected to the reactor, such that readings received from one or more sensors (14, 20-22) are conducted to the regulating element (17), where they are processed, on the basis of which processing the regulating element (17) controls one or more actuators (4, 7, 13, 16, 19), until a predetermined state is achieved.

23. An apparatus according to any of the claims 18-22, characterised in that the apparatus is complemented by a water tank (15) equipped with a pump (16), and a storage tank (18) containing concentrated binder solution, which storage tank is also equipped with a pump (19), and in that the pumps (16 and 19) are designed to pump water and/or concentrated binder solution into the reactor space (1) according to instructions provided by the regulating element (17).

24. An apparatus according to any of the claims 18-23, characterised in that the apparatus contains, before the reactor filter (3), going in the direction of flow of the gas, a gas flow atomising element (6), such as a superoxidant film or similar material, which forms essentially small microbubbles, and in that the reactor filler (3) is composed at least of a material, such as shavings or another sharp-edged material, that cuts the microbubbles and causes random vortices, which reactor filler (3) is pressed into the reactor space (1) to achieve a density greater than its original density.
